(19) United States
(12) Patent Application Publication
(10) Pub. No.: US 2012/0121586 A1
(43) Pub. Date: May 17, 2012

(54) MODULATORS FOR HER2 SIGNALING IN HER2 EXPRESSING PATIENTS WITH GASTRIC CANCER

(76) Inventors: Astrid Kiermaier, Loerrach (DE); Marlene Pickl, Penzberg (DE); Josef Rueschoff, Kassel (DE)

(21) Appl. No.: 13/319,869

(22) PCT Filed: May 28, 2010
(86) PCT No.: PCT/EP2010/057429

§ 371 Date: Nov. 10, 2011

(30) Foreign Application Priority Data
May 29, 2009 (EP) 09007217.4
Mar. 2, 2010 (EP) 10155170.3

(51) Int. Cl. A61K 39/395; A61P 35/00; C12Q 1/68

(52) U.S. Cl. 424/133.1; 435/6.11; 424/174.1

(57) ABSTRACT

The present invention relates to means and methods for the identification of responders for or a patient sensitive to a modulator of the HER2/neu (ErbB2) signaling pathway. Also described herein are corresponding methods of treatment of a group of patients determined and defined in accordance with the identification method of the present invention, whereby said group of patients is known or suspected to suffer from or being prone to suffer from gastric cancer in particular invasive gastric cancer.

The present invention relates to means and methods for the identification of responders for or a patient sensitive to a modulator of the HER2/neu (ErbB2) signaling pathway. Also described herein are corresponding methods of treatment of a group of patients determined and defined in accordance with the identification method of the present invention, whereby said group of patients is known or suspected to suffer from or being prone to suffer from gastric cancer, in particular invasive gastric cancer.

The members of the HER family of receptor tyrosine kinases are important mediators of cell growth, differentiation and survival. The receptor family includes four distinct members including epidermal growth factor receptor (EGFR, ErbB1, or HER1), HER2 (ErbB2 or p185^{neu}), HER3 (ErbB3) and HER4 (ErbB4). EGFR, encoded by the erbB1 gene, has been causally implicated in human malignancy. In particular, increased expression of EGFR has been observed in breast, bladder, lung, head, neck and stomach cancer as well as glioblastomas. Increased EGFR receptor expression is often associated with increased production of the EGFR ligand, transforming growth factor alpha (TGF-α), by the same tumor cells resulting in receptor activation by an autocrine stimulatory pathway. Baselga and Mendelsohn, Pharmac. Ther. 64:127-154 (1994). Monoclonal antibodies directed against the EGFR or its ligands, TGF-α and EGF, have been evaluated as therapeutic agents in the treatment of such malignancies. See, e.g., Baselga and Mendelsohn, supra; Masui et al. Cancer Research 44:1002-1007 (1984); and Wu et al. J. Clin. Invest. 95:1897-1905 (1995).

The second member of the HER family, p185^{neu}, was originally identified as the product of the transforming gene from neuroblastomas of chemically treated rats. Amplification of the human homolog of the neu gene (also known as HER2) is observed in breast and ovarian cancers and correlates with a poor prognosis (Slamon et al., Science, 235:177-182 (1987); Slamon et al., Science, 244:707-712 (1989); and US Pat No. 4,968,603). Overexpression of HER2 has also been observed in other carcinomas including carcinomas of the stomach, endometrium, salivary gland, lung, kidney, colon, thyroid, pancreas and bladder. See, among others, King et al., Science, 229:974 (1985); Yokota et al., Lancet: 1:765-767 (1986); Fukushige et al., Mol Cell Biol., 6:955-958 (1986); Guerin et al., Oncogene Res., 3:21-31 (1988); Cohen et al., Oncogene, 4:81-88 (1989); Yonemura et al., Cancer Res., 51:1034 (1991); Borst et al., Gynecol. Oncol., 38:364 (1990); Weiner et al., Cancer Res., 50:421-425 (1990); Kern et al., Cancer Res., 50:5184 (1990); Park et al., Cancer Res., 49:6605 (1989); Zhau et al., Mol. Carcinog., 3:254-257 (1990); Aasland et al. Br. J. Cancer 57:358-363 (1988); Williams et al. Pathobiology 59:46-52 (1991); and McCann et al., Cancer, 65:88-92 (1990). HER2 may be overexpressed in prostate cancer (Gu et al. Cancer Lett. 99:185-9 (1996); Ross et al. Hum. Pathol. 28:827-33 (1997); Ross et al. Cancer 79:2162-70 (1997); and Sadasivan et al. J. Urol. 150:126-31 (1993)). Antibodies directed against the rat p185^{neu} and human HER2 proteins have been described.

Drebin and colleagues have raised antibodies against the rat neu gene product, p185^{neu}; see, for example, Drebin et al., Cell 41:695-706 (1985); Myers et al., Meth. Enzym. 198:277-290 (1991); and W094/22478. Drebin et al. Oncogene 2:273-277 (1988) report that mixtures of antibodies reactive with two distinct regions of p185^{neu} result in synergistic anti-tumor effects on neu-transformed NIH-3T3 cells implanted into nude mice; see also U.S. Pat. No. 5,824,311 issued Oct. 20, 1998.

Hudziak et al., Mol. Cell. Biol. 9(3):1165-1172 (1989) describe the generation of a panel of HER2 antibodies which were characterized using the human breast tumor cell line SK-BR-3. Relative cell proliferation of the SK-BR-3 cells following exposure to the antibodies was determined by crystal violet staining of the monolayers after 72 hours. Using this assay, maximum inhibition was obtained with the antibody called 4D5 which inhibited cellular proliferation by 56%. Other antibodies in the panel reduced cellular proliferation to a lesser extent in this assay. The antibody 4D5 was further found to sensitize HER2-overexpressing breast tumor cell lines to the cytotoxic effects of TNF-α; see also U.S. Pat. No. 5,677,171 issued Oct. 14, 1997. The HER2 antibodies discussed in Hudziak et al. are further characterized in Fendly et al. Cancer Research 50:1550-1558 (1990); Kotts et al. In Vitro 26(3):59A (1990); Sarup et al. Growth Regulation 1:72-82 (1991); Shepard et al. J. Clin. Immunol. 11(3):117-127 (1991); Kumar et al. Mol. Cell. Biol. 11(2):979-986 (1991); Lewis et al. Cancer Immunol. Immunother. 37:255-263 (1993); Pietras et al. Oncogene 9:1829-1838 (1994); Vitetta et al. Cancer Research 54:5301-5309 (1994); Sliwkowski et al. J. Biol. Chem. 269(20):14661-14665 (1994); Scott et al. J. Biol. Chem. 266:14300-5 (1991); D'souza et al. Proc. Natl. Acad. Sci. 91:7202-7206 (1994); Lewis et al. Cancer Research 56:1457-1465 (1996); and Schaefer et al. Oncogene 15:1385-1394 (1997). A recombinant humanized version of the murine HER2 antibody 4D5 (huMAb4D5-8, rhuMAb HER2, Trastuzumab or HERCEPTIN®; U.S. Pat. No. 5,821,337) is clinically active in patients with HER2-overexpressing metastatic breast cancers that have received extensive prior anti-cancer therapy (Baselga et al., J. Clin. Oncol. 14:737-744 (1996)). Trastuzumab received marketing approval from the Food and Drug Administration Sep. 25, 1998 for the treatment of patients with metastatic breast cancer whose tumors overexpress the HER2 protein.

Other HER2 antibodies with various properties have been described in Tagliabue et al. Int. J. Cancer 47:933-937 (1991); McKenzie et al. Oncogene 4:543-548 (1989); Maier et al. Cancer Res. 51:5361-5369 (1991); Bacus et al. Molecular Carcinogenesis 3:350-362 (1990); Stancovski et al. PNAS (USA) 88:8691-8695 (1991); Bacus et al. Cancer Research 52:2580-2589 (1992); Xu et al. Int. J. Cancer 53:401-408 (1993); WO94/00136; Kasprzyk et al. Cancer Research 52:2771-2776 (1992); Hancock et al. Cancer Res. 51:4575-4580 (1991); Shawver et al. Cancer Res. 54:1367-1373 (1994); Arteaga et al. Cancer Res. 54:3758-3765 (1994); Harwerth et al. J. Biol. Chem. 267:15160-15167 (1992); U.S. Pat. No. 5,783,186; and Klapper et al. Oncogene 14:2099-2109 (1997).

Homology screening has resulted in the identification of two other HER family members; HER3 (U.S. Pat. Nos. 5,183,884 and 5,480,968 as well as Kraus et al. PNAS (USA) 86:9193-9197 (1989)) and HER4 (EP Pat Appln No 599,274; Plowman et al., Proc. Natl. Acad. Sci. USA, 90:1746-1750 (1993); and Plowman et al., Nature, 366:473-475 (1993)). Both of these receptors display increased expression on at least some breast cancer cell lines. The HER receptors are generally found in various combinations in cells and heterodimerization is thought to increase the diversity of cellular responses to a variety of HER ligands (Earp et al. Breast Cancer Research and Treatment 35: 115-132 (1995)). EGFR is bound by six different ligands; epidermal growth factor (EGF), transforming growth factor alpha (TGF-α), amphiregulin, heparin binding epidermal growth factor (HB-EGF), betacellulin and epiregulin (Groenen et al. Growth Factors 11:235-257 (1994)). A family of heregulin proteins resulting from alternative splicing of a single gene are ligands for HER3 and HER4. The heregulin family includes alpha, beta and gamma heregulins (Holmes et al., Science, 256:1205-1210 (1992); U.S. Pat. No. 5,641,869; and Schaefer et al. Oncogene 15:1385-1394 (1997)); neu differentiation factors (NDFs), glial growth factors (GGFs); acetylcholine receptor inducing activity (ARIA); and sensory and motor neuron derived factor (SMDF). For a review, see Groenen et al. Growth Factors 11:235-257 (1994); Lemke, G. Molec. & Cell. Neurosci. 7:247-262 (1996) and Lee et al. Pharm. Rev. 47:51-85 (1995). Recently three additional HER ligands were identified; neuregulin-2 (NRG-2) which is reported to bind either HER3 or HER4 (Chang et al. Nature 387 509-512 (1997); and Carraway et al Nature 387:512-516 (1997)); neuregulin-3 which binds HER4 (Zhang et al. PNAS (USA) 94(18):9562-7 (1997)); and neuregulin-4 which binds HER4 (Harari et al. Oncogene 18:2681-89 (1999)) HB-EGF, betacellulin and epiregulin also bind to HER4.

While EGF and TGFα do not bind HER2, EGF stimulates EGFR to form a heterodimer with HER2, which results in transphosphorylation of HER2 by EGFR and vice versa in the heterodimer; see Earp et al., supra. Likewise, when HER3 is co-expressed with HER2, an active signaling complex is formed and antibodies directed against HER2 are capable of disrupting this complex (Sliwkowski et al., J. Biol. Chem., 269(20):14661-14665 (1994)). Additionally, the affinity of HER3 for heregulin (HRG) is increased to a higher affinity state when co-expressed with HER2. See also, Levi et al., Journal of Neuroscience 15:1329-1340 (1995); Morrissey et al., Proc. Natl. Acad. Sci. USA 92: 1431-1435 (1995); and Lewis et al., Cancer Res., 56:1457-1465 (1996) with respect to the HER2-HER3 protein complex. HER4, like HER3, forms an active signaling complex with HER2 (Carraway and Cantley, Cell 78:5-8 (1994)). Patent publications related to HER antibodies include: U.S. Pat. No. 5,677,171, U.S. Pat. No. 5,720,937, U.S. Pat. No. 5,720,954, U.S. Pat. No. 5,725,856, U.S. Pat. No. 5,770,195, U.S. Pat. No. 5,772,997, U.S. Pat. No. 6,165,464, U.S. Pat. No. 6,387,371, U.S. Pat. No. 6,399,063, US2002/0192211A1, U.S. Pat. No. 6,015,567, U.S. Pat. No. 6,333,169, U.S. Pat. No. 4,968,603, U.S. Pat. No. 5,821,337, U.S. Pat. No. 6,054,297, U.S. Pat. No. 6,407,213, U.S. Pat. No. 6,719,971, U.S. Pat. No. 6,800,738, US2004/0236078A1, U.S. Pat. No. 5,648,237, U.S. Pat. No. 6,267,958, U.S. Pat. No. 6,685,940, U.S. Pat. No. 6,821,515, WO98/17797, U.S. Pat. No. 6,127,526, U.S. Pat. No. 6,333,398, U.S. Pat. No. 6,797,814, U.S. Pat. No. 6,339,142, U.S. Pat. No. 6,417,335, U.S. Pat. No. 6,489,447, WO99/31140, US2003/0147884A1, US2003/0170234A1, US2005/0002928A1, U.S. Pat. No. 6,573,043, US2003/0152987A1, WO99/48527, US2002/0141993A1, WO01/00245, US2003/0086924, US2004/0013667A1, WO00/69460, WO01/00238, WO01/15730, U.S. Pat. No. 6,627,196B1, U.S. Pat. No. 6,632,979B1, WO01/00244, US2002/0090662A1, WO01/89566, US2002/0064785, US2003/0134344, WO 04/24866, US2004/0082047, US2003/0175845A1, WO03/087131, US2003/0228663, WO2004/008099A2, US2004/0106161, WO2004/048525, US2004/0258685A1, U.S. Pat. No. 5,985,553, U.S. Pat. No. 5,747,261, U.S. Pat. No. 4,935,341, U.S. Pat. No. 5,401,638, U.S. Pat. No. 5,604,107, WO 87/07646, WO 89/10412, WO 91/05264, EP 412,116 B1, EP 494,135 B1, U.S. Pat. No. 5,824,311, EP 444,181 B1, EP 1,006,194 A2, US 2002/0155527A1, WO 91/02062, U.S. Pat. No. 5,571,894, U.S. Pat. No. 5,939,531, EP 502,812 B1, WO 93/03741, EP 554,441 B1, EP 656,367 A1, U.S. Pat. No. 5,288,477, U.S. Pat. No. 5,514,554, U.S. Pat. No. 5,587,458, WO 93/12220, WO 93/16185, U.S. Pat. No. 5,877,305, WO 93/21319, WO 93/21232, U.S. Pat. No. 5,856,089, WO 94/22478, U.S. Pat. No. 5,910,486, U.S. Pat. No. 6,028,059, WO 96/07321, U.S. Pat. No. 5,804,396, U.S. Pat. No. 5,846,749, EP 711,565, WO 96/16673, U.S. Pat. No. 5,783,404, U.S. Pat. No. 5,977,322, U.S. Pat. No. 6,512,097, WO 97/00271, U.S. Pat. No. 6,270,765, U.S. Pat. No. 6,395,272, U.S. Pat. No. 5,837,243, WO 96/40789, U.S. Pat. No. 5,783,186, U.S. Pat. No. 6,458,356, WO 97/20858, WO 97/38731, U.S. Pat. No. 6,214,388, U.S. Pat. No. 5,925,519, WO 98/02463, U.S. Pat. No. 5,922,845, WO 98/18489, WO 98/33914, U.S. Pat. No. 5,994,071, WO 98/45479, U.S. Pat. No. 6,358,682 B 1, US 2003/0059790, WO 99/55367, WO 01/20033, US 2002/0076695 A1, WO 00/78347, WO 01/09187, WO 01/21192, WO 01/32155, WO 01/53354, WO 01/56604, WO 01/76630, W002/05791, WO 02/11677, U.S. Pat. No. 6,582,919, US2002/0192652A1, US 2003/0211530A1, WO 02/44413, US 2002/0142328, U.S. Pat. No. 6,602,670 B2, WO 02/45653, WO 02/055106, US 2003/0152572, US 2003/0165840, WO 02/087619, WO 03/006509, WO03/012072, WO 03/028638, US 2003/0068318, WO 03/041736, EP 1,357,132, US 2003/0202973, US 2004/0138160, U.S. Pat. No. 5,705,157, U.S. Pat. No. 6,123,939, EP 616,812 B1, US 2003/0103973, US 2003/0108545, U.S. Pat. No. 6,403,630 B1, WO 00/61145, WO 00/61185, U.S. Pat. No. 6,333,348 B1, WO 01/05425, WO 01/64246, US 2003/0022918, US 2002/0051785 A1, U.S. Pat. No. 6,767,541, WO 01/76586, US 2003/0144252, WO 01/87336, US 2002/0031515 A1, WO 01/87334, WO 02/05791, WO 02/09754, US 2003/0157097, US 2002/0076408, WO 02/055106, WO 02/070008, WO 02/089842 and WO 03/86467.

Breast cancer patients treated with the HER2 antibody Trastuzumab are selected for therapy based on HER2 overexpression/amplification; see, for example, WO99/31140 (Paton et al.), US2003/0170234A1 (Hellmann, S.), and US2003/0147884 (Paton et al.); as well as WO01/89566, US2002/0064785, and US2003/0134344 (Mass et al.).

The prior art was focused on the eligibility of breast cancer patients for Trastuzumab/Herceptin therapy based on a high HER2 protein expression level (e.g. HER2(3+) by IHC). Yet, little information is available on the eligibility of gastric cancer patients for such an antibody therapy. There is also dispute in the art whether the scoring system and the criteria developed for screening and identifying breast cancer patients which might respond to antibody therapy are applicable to other cancer types, in particular gastric cancer which show a quite different histopathological pattern and concern overall a different pathology.

Methods for detecting HER2 overexpression and amplification by immunohistochemistry (IHC) and fluorescence in situ hybridization (FISH) are also described in US2003/0152987, Cohen et al.

WO2004/053497 and US2004/024815A1 (Bacus et al.), as well as US 2003/0190689 (Crosby and Smith), refer to determining or predicting response to Trastuzumab therapy. US2004/013297A1 (Bacus et al.) concerns determining or predicting response to ABX0303 EGFR antibody therapy. WO2004/000094 (Bacus et al.) is directed to determining response to GW572016, a small molecule, EGFR-HER2 tyrosine kinase inhibitor. WO2004/063709, Amler et al., refers to biomarkers and methods for determining sensitivity to EGFR inhibitor, erlotinib HCl. US2004/0209290, Cobleigh et al., concerns gene expression markers for breast cancer prognosis. Breast cancer patients to be treated with Pertuzumab (a HER2 dimerisation inhibitor described herein below in more detail) can be selected for therapy based on HER overexpression/amplification, activation or dimerization. Patent publications concerning pertuzumab and selection of patients for therapy therewith include: WO01/00245 (Adams et al.); US2003/0086924 (Sliwkowski, M.); US2004/0013667A1 (Sliwkowski, M.); as well as WO2004/008099A2, and US2004/0106161(Bossenmaier et al.).

Pertuzumab (formerly 2C4) is the first of a new class of agents known as HER dimerisation inhibitors (HDIs). Pertuzumab binds to HER2 at its dimerization domain, thereby inhibiting its ability to form active dimer receptor complexes and thus blocking the downstream signal cascade that ultimately results in cell growth and division; see Franklin (2004), Cancer Cell 5, 317-328. Pertuzumab is a fully humanized recombinant monoclonal antibody directed against the extracellular domain of HER2. Binding of Pertuzumab to the HER2 on human epithelial cells prevents HER2 from forming complexes with other members of the HER family (including EGFR, HER3, HER4) and probably also HER2 homodimerization. By blocking complex formation, Pertuzumab prevents the growth stimulatory effects and cell survival signals activated by ligands of HER1, HER3 and HER4 (e.g. EGF, TGFα, amphiregulin, and the heregulins). Another name for Pertuzumab is 2C4. Pertuzumab is a fully humanized recombinant monoclonal antibody based on the human IgG1(κ) framework sequences. The structure of Pertuzumab consists of two heavy chains (449 residues) and two light chains (214 residues). Compared to Trastuzumab (Herceptin®), Pertuzumab has 12 amino acid differences in the light chain and 29 amino acid differences in the IgG1 heavy chain.

Herceptin is widely used and known in the art for the treatment of patients with early as welt as metastatic breast cancer whose tumors overexpress HER2 protein or have HER 2 gene amplification. In the art, the treatment of breast cancer patients with Herceptin/Trastuzumab is, for example, recommended and routine for patients having HER2-positive disease. HER2-positive disease in breast cancer is present if a high HER2 (protein) expression level detected by immunohistochemical methods (e.g. HER2 (+++) or HER2 gene amplification (e.g. a HER2 gene copy number higher than 4 copies of the HER2 gene per tumor cell) or both is found in samples obtained from the patients such as breast tissue biopsies or breast tissue resections or in tissue derived from metastatic sites.

However, it is not known whether gastric cancer patients having an equivocal or high HER2 protein expression level e.g. HER2(2+) or HER2(3+) can successfully be treated with Herceptin and whether the amplification status of HER2 might indicate a sensitivity to the treatment of gastric cancer.

Thus, the technical problem underlying the present invention is the provision of means and methods of identification of a patient or a group of patients suffering from or being prone to suffer from gastric cancer who may be responsive to a treatment of gastric cancer with a modulator of the HER2/neu (ErbB2) signaling pathway, in particular to a treatment with a HER2 antibody such as Trastuzumab/Herceptin.

The technical problem is solved by provision of the embodiments characterized in the claims.

Accordingly, the present invention relates to an in vitro method for the identification of a patient suspected to suffer from gastric cancer as a responder for or a patient sensitive to a modulator of the HER2/neu (ErbB2) signaling pathway, said method comprising the following steps:

(a) obtaining a sample from said patient; and

(b) evaluating

(b1) the expression level of HER2 protein in said sample; and

(b2) the gene amplification status of the HER2 gene in said sample;

whereby an equivocal expression level of HER2 protein and a high gene amplification status of the HER2 gene is indicative for a responding patient or is indicative for a sensitivity of said patient to said modulator of the HER2/neu (ErbB2) signaling pathway.

It is preferred in context of the present invention that the expression level of HER2 protein is detected by an immunohistochemical method, whereas said HER2 gene amplification status can be measured with in situ hybridization (ISH) methods, like fluorescence in situ hybridization techniques (FISH), chromogenic in situ hybridization techniques (CISH) or silver in situ hybridization techniques (SISH). Corresponding assays and kits are well known in the art, for protein expression assays as well as for the detection of gene amplifications.

The present invention solves the above identified technical problem since, as described herein below, it was surprisingly found that an unexpected group of patients that is characterized by an equivocal expression level of HER2 protein, i.e. HER2 (2+) as, e.g., determined by IHC, and a high gene amplification status of the HER2 gene (e.g. an average copy number of the HER2 gene higher than 4 per tumour cell or an average gene copy number of the HER2 gene equal to or higher than 2 per chromosome 17 copy (per tumour cell)) is responsive to a treatment with a modulator of the HER2/neu (erbB2) signaling pathway, in particular with a HER2 antibody, like Herceptin/Trastuzumab.

The term “average” as used herein, e.g., in context with an average copy number of the HER2 gene, relates to the average number of HER2 gene copies per tumour cells of at least two tumour cells of a biological sample to be examined or to the average number of HER2 gene copies per chromosome 17 copy in at least two tumour cells of a biological sample to be examined.

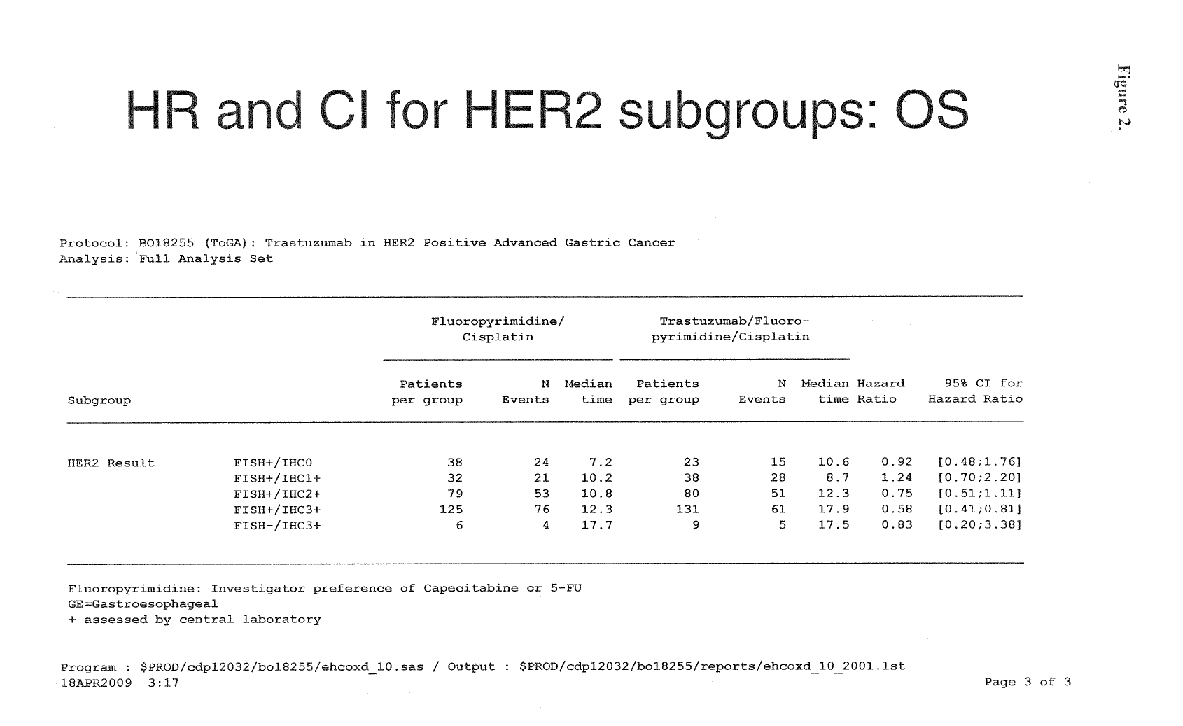

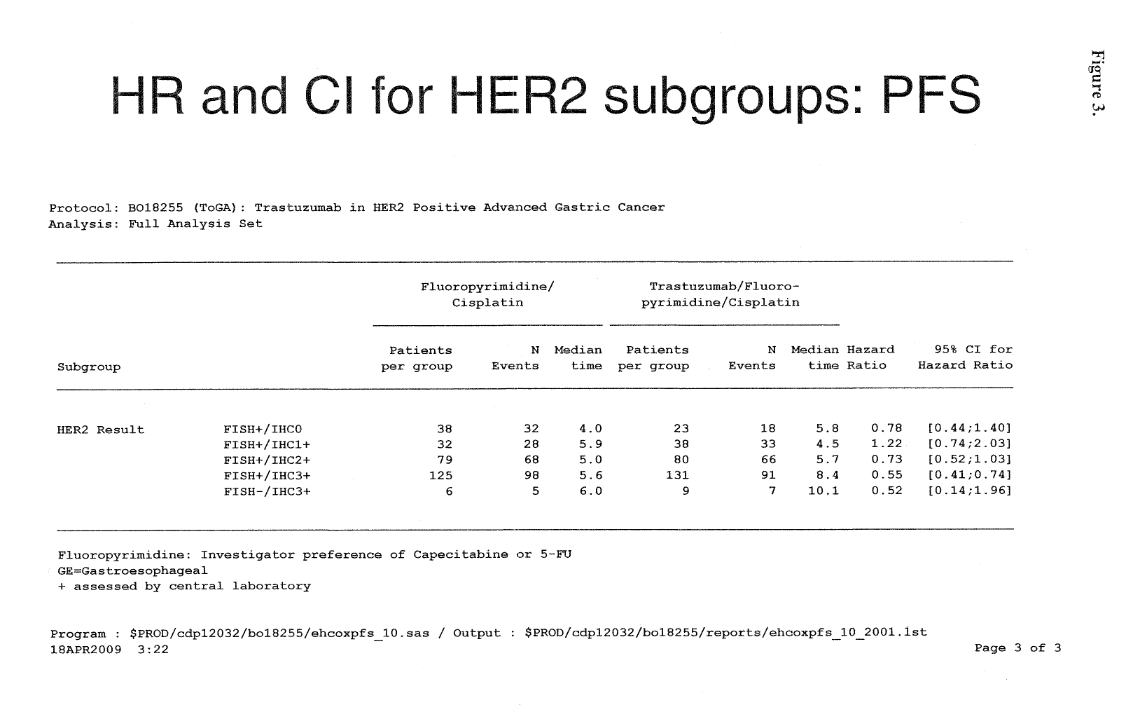

In the present invention it was surprisingly found that gastric cancer patients which have both an equivocal level of HER2 protein expression in a biological sample (e.g. biopsies or resectats) and at the same time a high amplification of the HER2 gene in a biological sample (e.g. biopsies or resectats) are responsive to the treatment with such a HER2 antibody. Unexpectedly, the response rate of these patients to a treatment with a modulator of the HER2/neu (erbB2) signaling pathway is by far higher compared to gastric cancer patients having a high amplification of the HER2 gene but only low or moderate staining in IHC; see the appended example and figures, in particular FIGS. 2 and 3. The novel patient group identified in accordance with the present invention shows a better response to treatment with a HER2 modulator compared to the low HER2 protein expressing group (e.g. IHC(0) or IHC(1+) by IHC) which is, in addition, characterized by a high HER2 gene amplification.

In the art there is no clear-cut recommendation on how to stratify gastric cancer patients for treatment with a HER2 modulator. Samples obtained from patients suffering from gastric cancer which are evaluated as equivocally expressing HER2 protein may or may not be further investigated by ISH. In the art there is only data on incidence of HER2 overexpression and/or gene amplification in gastric cancer but no data on predictivity of HER2 status with regard to treatment benefit with a HER2 modulator neither in relation to the HER2 protein level nor in relation to the HER2 gene level. Hence, the prior art does not suggest further testing of the amplification status for samples showing an equivocal or intermediate level of HER2 protein expression. ISH testing of the HER2 gene amplification status is not performed on a routinely basis in gastric cancer patients. The prior art does not suggest further testing of the amplification status of HER2 gene after determination and finding of an intermediate or equivocal expression level of HER2 protein in gastric cancer samples.

Surprisingly, it was found herein that patients with an equivocal expression level of HER2 protein and a high gene amplification status of the HER2 gene can successfully be treated with a modulator of the HER2/neu (ErbB2) signaling pathway. Moreover, a patient/ the patient group to be identified and treated in accordance with the present invention is preferably characterized by an equivocal expression level of HER2 protein (e.g. a “2+” score as defined herein, i.e. HER2(2+) determined by IHC (equivocal assessment, see the tables below) having at the same time a high amplification status of the HER2 gene. As illustrated in the appended Example, this patient group with an equivocal expression level of HER2 protein (e.g. HER2(2+) determined by IHC) and a high amplification status of the HER2 gene as defined herein (e.g. copy number of the HER2 gene of higher than 4 or a gene copy number of HER2 equal to or higher than 2 per chromosome 17 copy) is surprisingly more responsive/shows a higher sensitivity to a modulator of the HER2/neu (ErbB2) signaling pathway, in particular to Herceptin/Trastuzumab than patients with a low HER2 expression level of HER 2 protein (e.g. a “0” or “1+” score as defined herein, i.e. HER2(0) or HER2(1+)) and high HER2 gene amplification.

The methods according to the present invention amongst other aspects are based on the surprising finding that testing a patient sample first for HER2 gene amplification may lead to false positives. As can be seen from the results shown, patients with HER2 gene amplification in the absence of a equivocal HER2 protein expression level do not respond well the Herceptin/Trastuzumab treatment. This finding in gastric cancer is the more surprising since the response to a HER2 modulator in breast cancer is generally known to correlate with the amplification level of HER2 gene independent of the HER2 protein level as detected by IHC. An even superior response to a HER2 modulator in patients with gastric cancer as compared to standard therapy (e.g. flouropyrimidine/cisplatin) was certainly not known in the prior art. Moreover, an at least comparable or even better response of the equivocally HER2 gene expressing subgroup with a high amplification of the HER2 gene as compared to the low expressing HER2 protein group also having an amplification of the HER2 gene was an unexpected finding in gastric cancer treatment.

Accordingly, the present invention relates in one embodiment to an in vitro method for the identification of a patient suspected to suffer from gastric cancer and having an equivocal expression of HER2 protein as a responder for or a patient sensitive to a modulator of the HER2/neu (ErbB2) signaling pathway, said method comprising the following steps:

(a) obtaining a sample from said patient; and

(b) evaluating

the gene amplification status of the HER2 gene in said sample,
whereby an equivocal expression level of HER2 protein and a high gene amplification status of the HER2 gene is indicative for a responding patient or is indicative for a sensitivity of said patient to said modulator of the HER2/neu (ErbB2) signaling pathway.

As also documented in the appended Example, not only the patient/patient group having a “HER2(2+)” score in IHC as defined herein and at the same time amplification of the HER2 gene, but also the patient group with a “3+” IHC score can successfully be treated and is responsive to/shows a high sensitivity to a modulator of the HER2/neu (ErbB2) signaling pathway, in particular to Herceptin/Trastuzumab. The patient group having a high level of HER2 protein expression (i.e. IHC (3+) is also responsive for treatment with a HER2 modulator and should be treated irrespective of its gene amplification status. Even patients not showing a gene amplification but tested highly positive for HER2 protein expression (IHC 3+) show a good response to treatment (see FIGS. 2 and 3).

The successive testing by HER2 IHC and by HER2 ISH, in case of an equivocal HER2 IHC result (e.g., HER2(2+) as measured for example by IHC), also represents a preferred embodiment of the present invention. In other words, the method of the present invention may comprise in a second step evaluating the amplification status of HER2 gene subsequent to the evaluation of the expression level of HER2 protein in a first step, only if the expression level of HER2 protein assessed in the first step is equivocal (e.g. HER2(2+) by IHC) As mentioned, overexpression of the HER2 protein, e.g. as measured by IHC, or amplification of the HER2 gene, e.g. as determined in an ISH method, represents independent and equally valuable eligibility criteria for HER2 modulator treatment in breast cancer. However and surprisingly, this is not true for patients with gastric cancer.

Accordingly, the present invention relates in a further embodiment to an in vitro method for the identification of a responder for or a patient sensitive to a modulator of the HER2/neu (ErbB2) signaling pathway, said method comprising the following steps:

(a) obtaining a sample from a patient suspected to suffer from or being prone to suffer from gastric cancer; and

(b) evaluating

(b1) in a first step the expression level of HER2 protein; and

(b2) in a second step the gene amplification status of the HER2 gene in said sample, if the expression level of HER2 protein evaluated in the first step is equivocal;

whereby an equivocal expression level of HER2 protein and a high gene amplification status of the HER2 gene is indicative for a responding patient or is indicative for a sensitivity of said patient to said modulator of the HER2/neu (ErbB2) signaling pathway

The level for HER2 protein or gene expression can be assessed either on the protein or on the nucleic acid level. The HER2 protein expression level is preferably evaluated using immunohistological methods, like “IHC” (immunohistochemistry), see also details herein. The HER2 gene amplification can be assessed by further methods known in the art, which comprise, but are not limited to the determination of the average HER2 gene copy number in cells of a given sample or the determination of the HER2/CEP17 ratio. Details on representative methods are provided herein below.

In accordance with this invention, the expression level of HER2 protein is preferably measured by immunohistochemical (IHC) methods employing antibodies against the HER2 protein, whereas the HER2 gene amplification in biological samples is detected by in situ hybridization (ISH) method, like preferably FISH, CISH or SISH. As described herein, a patient group with an equivocal expression of HER2 protein (e.g. HER2(2+)) and a high gene amplification status of HER2 gene (e.g. a(n) (average) copy number of higher than 4) has been identified as being responsive to a modulator of the HER2/neu signaling pathway. It is also envisaged and preferred herein that a patient with an equivocal expression of HER2 protein (e.g. HER2(2+)) and a high gene amplification status of HER2 gene (e.g. a(n) (average) copy number of higher than 4) is sensitive to said modulator.

Patients with an equivocal expression of HER2 protein (e.g. HER2(2+)) and high amplification status of the HER2 gene can easily be separated from patients with low expression of HER2 protein (e.g. HER2(0) or HER2(1+))), since a person skilled in the art is aware of standard tests, in particular of immunohistochemical tests, for such a determination of the expression level of HER2 protein. This means that a sub-group of the large patient group showing an equivocal HER2 protein expression level can be successfully subjected to treatment with a HER2 antibody, i.e. the patients having an equivocal HER2 protein expression and high amplification status of the HER2 gene. As described herein below in more detail, a successful treatment of gastric cancer patients, in particular invasive gastric cancer patients, having an equivocal HER2 protein expression level has neither been described nor proposed in the state of the art. To the contrary, a person skilled in the art would not have believed that certain individuals out of the isolated patient group with an equivocal HER2 protein expression level can be more successfully treated, if these patients have a high amplification status of the HER2 gene.

In breast cancer various different patient inclusion algorithms are known either based on the protein expression level of HER2, the gene amplification status of HER2 or both (alone or in combination), wherein it is generally believed that the higher the expression level of HER2, the more likely will a breast cancer patient respond to a treatment. However, gastric cancer differs in various aspects from breast cancer and nothing is known about the utility of the amplification status of the HER2 gene as an eligibility criterion in gastric cancer.

In contrast to the situation for breast cancer as known in the art, the results of the study presented herein (see FIGS. 2 and 3) clearly show that patients having an equivocal HER2 protein (HER2(2+)) expression level and a high amplification of the HER2 gene can, unexpectedly, successfully be treated, whereas patients with low expression of HER2 protein (i.e. HER2(0) or HER2(1+)) appear not to benefit from treatment with a HER2 modulator even if the HER2 gene is amplified.

The present invention describes for the first time that gastric cancer patients, in particular invasive gastric cancer patients, having an equivocal HER2 protein expression level (e.g. HER2(2+) in immunohistochemical detection of HER2 protein in a biological sample such as a gastric cancer cell/tissue) and high amplification of the HER2 gene in a biological sample can successfully be treated with a modulator of the HER2/neu (ErbB2) signaling pathway. Particularly useful are therapeutic antibodies, e.g., an HER2 antibody such as Herceptin/Trastuzumab. The group of patients identified by the method of the present application surprisingly shows a response rate to HER2 treatment which is higher than the response rate in patients having low level of HER2 protein expression (e.g. HER2((0) or HER2(1+) by IHC) and a high amplification of the HER2 gene. As a consequence, a sub-group of patients having an equivocal expression level of HER2 protein can be subject to successful treatment with a modulator of the HER2/neu (erbB2) signaling pathway, thus increasing the survival rate and the progression free survival of these patients. The finding of the present invention therefore represents a major contribution to the art and provides an important benefit to many gastric cancer patients, in particular invasive gastric cancer patients. Furthermore, the present invention relates to a method of treatment of patients having an equivocal level of expression of HER2 protein (HER2(2+)) and highly amplified HER2 genes and corresponding means and methods.

The terms “responder for a modulator of the HER2/neu (ErbB2) signaling pathway” means in the context of the present invention that a subject/patient suspected to suffer from or being prone to suffer from gastric cancer shows a response to a treatment with the modulator. An artisan will readily be in the position to determine whether a person treated with the modulator shows a response. For example, a response to a modulator may be reflected in a decreased suffering from gastric cancer, such as a diminished and/or halted growth of a gastric cancer tumor and/or a reduction of the size of a tumor, the prevention of the formation of metastases or a reduction of number or size of metastases. It is preferred that a response is reflected in the prevention of the development of a gastric cancer tumor or metastases, for example after resection of a tumor in the prolongation of time to disease progression, or in the reduction of the size of (a) tumor(s) and/or (a) metastases, for example in anti-metastatic or neoadjuvant therapy.

Similarly, the term “patient sensitive to a modulator of the HER2/neu (ErbB2) signaling pathway” refers in the context of the present invention to a patient which shows in some way a positive reaction when treated with the modulator. This reaction of the patient may be less pronounced when compared to a responder as described herein above. For example, the patient may experience less suffering from gastric cancer though no reduction in tumor growth may be measured. The reaction of the patient to the modulator may also be only of a transient nature, i.e. growth of (a) tumor and/or (a) metastasis(es) may only be temporarily reduced or halted. It is preferred that a responder for a modulator will not suffer from gastric cancer after treatment with the modulator. Preferably, (a) gastric cancer tumor(s) and/or (a) gastric cancer metastasis(es) which has been treated with a HER2 modulator will not recur within 1 year after termination of the treatment of the responder with the modulator, also, preferably, not within 15 months, 18 months or 2 years after termination of the treatment.

The term “gastric cancer” as used in the present invention relates to all types of gastric cancer including gastro-esophageal junction (GEJ) carcinomas. In one preferred embodiment, the gastric cancer in the sense of the present invention relates to inoperable cancer, e.g. to locally advanced or recurrent and/or metastatic cancer of the stomach or the gastro-oesophageal junction.

The term “anti-metastatic” treatment relates to the treatment of a patient suffering from an inoperable tumour or an advanced tumour without performing a surgery.

As the skilled artisan fully appreciates, a positive test for HER2 gene amplification in the HER2 protein equivocal subgroup according to this invention does not translate 1:1 into a successful treatment. By these methods sub-groups of patients are identified that have a higher chance of response to a treatment with a HER2 signaling inhibitor as compared to the sub-groups of patients not showing these positive test results. Improvements in response to treatment are e.g. seen with respect to response rate, to shrinkage of tumours, to progression free survival (PFS) as well as with respect to overall survival (OS). The improvement in response of the isolated group of patients identified by the method of the present invention to a modulator of the HER2/neu (ErbB2) signaling pathway is at least 15%. Also preferred, the improvement in response is at least, 18%, or at least 20%. Also preferred, the improvement in response is at least 25% or 30%. With other words, a positive result indicates (=is indicative for) that the patient has a higher chance (=probability, likelihood) to respond to (=of being susceptible to) treatment with a HER2 signaling inhibitor as compared to a patient having, for example, low HER2 protein (preferably assessed by IHC) and high HER2 gene amplification. In one embodiment the improvement in response relates to a better response rate. In other embodiments the improvement in response relates to PFS or OS, respectively, as e.g. evident from FIGS. 2 and 3, respectively.

As mentioned above, it is preferred herein that the expression level of HER2 protein is detected by an immunohistochemical method. Such methods are well known in the art and corresponding commercial kits are easily available. Exemplary kits which may be used in accordance with the present invention are, inter alia, HerceptTest™, produced and distributed by the company Dako, Denmark, or the test called Ventana Pathway™, produced and distributed by Ventana, Tucson, USA. Preferably the level of HER2 protein expression is assessed by using the reagents provided with and following the protocol of the HercepTest™. A skilled person will be aware of further means and methods for determining the expression level of HER2 protein by immunohistochemical methods; see for example WO 2005/117553. Therefore, the expression level of HER2 protein can be easily and reproducibly determined by a person skilled in the art without undue burden. However, to ensure accurate and reproducible results, the testing should be performed in a specialized laboratory, which can ensure validation of the testing procedures.

The expression level of HER2 protein in gastric cancer can be classified in a low expression level, an equivocal expression level and a high expression level. It is preferred in context of this invention that a sample obtained from a patient suspected to suffer from or being prone to suffer from gastric cancer shows an equivocal expression level of HER2 protein.

Most preferably the equivocal protein expression level is HER2(2+). Furthermore, as used herein, the low protein expression level is HER2 (0/1+) and the high protein expression level is HER2(3+).

It is a new and surprising finding that different criteria should be applied to a resection sample and to a biopsy sample.

In context with the present invention, different scoring systems are applied for resection samples as compared to biopsy samples.

In one embodiment of the present invention, the scoring of a low, an equivocal or a high protein expression level of HER2 (e.g., HER2(0/1+), HER2(2+) or HER2(3+), respectively) is determined in a resection sample and scoring is based on the criteria of the table below.

| Score | Staining Pattern (resection sample) | classification |
|---|---|---|
| 0 | No reactivity or membranous reactivity | negative |
| | in <10% of cells | |
| 1+ | Faint/barely perceptible membranous | negative |
| | reactivity in >10% of cells; cells are | |
| | reactive only in part of their membrane | |
| 2+ | Weak to moderate complete or basolateral | equivocal |
| | membranous reactivity in >10% of tumour cells | |
| 3+ | Moderate to strong complete or basolateral | positive |
| | membranous reactivity in >10% of tumour cells | |

A gastric cancer resection sample is also considered positive if the resection sample shows a high protein expression level (e.g. IHC(3+) by IHC) in an area covering less than 10% of the tumour area, in particular if the IHC(3+) clones in this sample are cohesive.

In another embodiment of the present invention, the scoring of a low, an equivocal or a high protein expression level of HER2 (e.g., HER2(0/1+), HER2(2+) or HER2(3+), respectively) is determined in a biopsy sample and scoring is based on the criteria of the table below.

| Score | Staining pattern (biopsy sample) | classification |
|---|---|---|
| 0 | No reactivity or no membranous reactivity | Negative |
| | in any tumour cell | |
| 1+ | Tumour cell cluster with a faint/barely | Negative |
| | perceptible membranous reactivity irrespective | |
| | of percentage of tumour cells stained | |
| 2+ | Tumour cell cluster with a weak to moderate | Equivocal |
| | complete, basolateral or lateral membranous | |
| | reactivity irrespective of percentage of | |
| | tumour cells stained | |
| 3+ | Tumour cell cluster with a strong complete, | Positive |
| | basolateral or lateral membranous reactivity | |
| | irrespective of percentage of tumour cells stained | |

Preferably, the biopsy sample comprises at least five stained tumor cells. The at least five tumour cells are preferably cohesive tumour cells. Intermediate or equivocal staining is acknowledged if a tumour cell cluster with a weak to moderate complete, basolateral or lateral membranous staining is present in a biopsy specimen.

The terms HER2(+), HER2(++) and HER2(+++) used herein are equivalent to the terms HER2(1+), HER2(2+) and HER2(3+). A “low protein expression level” used in context of this invention corresponds to a “0” or “1+” score (“negative assessment” according to the tables shown herein above), an “equivocal protein expression level” corresponds to a “2+ “score and a “high protein expression level” corresponds to a “3+” score. As described hereinabove in detail, the evaluation of the protein expression level (i.e. the scoring system as shown in the tables) is based on results obtained by immunohistochemical methods. As a standard or routinely, the HER-2 status is, accordingly, performed by immunohistochemistry with one of two FDA-approved commercial kits available; namely the Dako Herceptest™ and the Ventana Pathway™, respectively. These are semi-quantitative assays which stratify expression levels into 0 (<20,000 receptors per cell, no expression visible by IHC staining), 1+ (˜100,000 receptors per cell, partial membrane staining, <10% of cells overexpressing HER2), 2+ (˜500,000 receptors per cell, light to moderate complete membrane staining, >10% of cells overexpressing HER2), and 3+ (˜2,000,000 receptors per cell, strong complete membrane staining, >10% of cells overexpressing HER2).

Alternatively, further methods for the evaluation of the protein expression level of HER2 may be used, e.g. Western Blots, ELISA-based detection systems and so on. An equivocal or high expression level of HER2 protein can be determined by these techniques and a biological sample of those patients classified as having an equivocal level of HER2 protein expression may be further analyzed for HER2 gene amplification.

As pointed out herein, the group of patients identified by the method of the invention and sensitive to treatment is characterized by an “equivocal” HER2 protein expression and, in addition, by a high HER2 gene amplification. Just for completeness of information it shall be mentioned that the group of patients identified as having a “high” HER2 expression is also sensitive to treatment with a modulator of HER2-signaling.

A high HER2 gene amplification status may, inter alia, relate to a gene copy number of the HER2 gene of higher than 4, in particular an average HER2 gene copy number higher than 4 copies of the HER2 gene per tumor cell (for those test systems without an internal centromere control probe) or to an average gene copy number of the HER2 gene equal to or higher than 2 per chromosome 17 copy (per tumor cell), in other words to a HER2/CEP17 ratio of higher than 2 (for those test systems using an internal chromosome 17 centromere control probe) (per tumor cell). The high HER2 gene amplification status preferably also relates to a HTR2 gene copy number of at least 5, 6, 7, 8 or higher or to a HER2/CEP17 ratio of at least 3, 4, 5 or 6. A HER2 gene copy number of 5 may, for example, arise from a duplication of 2 copies of the HER2 gene (e.g. duplication of two genes on two chromosomes or by duplication of the chromosomes carrying a copy of the HER2 gene), and an additional duplication of one copy of the HER2 gene within the same chromosome. Preferably, a sample shows a high amplification status of the HER2 gene in an area covering more than 10% of the tumour area. For example, cells with a high amplification status as defined herein cover more than 10% of the tumour area/cells assessed in accordance with the present invention. These tumour cells with a high amplification status may also be cohesive.

In a preferred embodiment of the present invention, the amplification status of HER2 gene is evaluated by in situ hybridization (ISH). Preferably, the in situ hybridization is fluorescent in situ hybridization (FISH), chromogenic in situ hybridization (CISH) and silver in situ hybridization (SISH). These methods are known to the skilled artisan. The principles of these methods can be deduced from standard text books. Commercial kits for the determination of the HER2 amplification status by in situ hybridization are easily available. Preferred FISH tests to be employed in accordance with the present invention are “Inform®” kit from Ventana, “Pathvysion™” kit from Abbott and “pharmDx™” kit from Dako. Preferred CISH assays are SPoT-Light® HER2 CISH™ from Invitrogen and ZytoDot® SPEC HER2 Probe kit from Zytovision. A preferred SISH-assay is the Inform™ HER2 DNA probe from Ventana in combination with the ultraView™ SISH detection kit from Ventana.

The HER2/neu (ErbB2) signaling pathway is well known in the art and a skilled person is readily in the position to identify such modulators based on his general knowledge and the teaching provided herein. Non-limiting examples of modulators to be used in accordance of this invention are antibodies, preferably monoclonal or humanized antibodies, like Herceptin/Trastuzumab or pertuzumab (see, e.g. WO2007/145862). A preferred embodiment according to this invention is the administration of Herceptin/Trastuzumab to the sub-group of gasstric cancer patients characterized by an equivocal protein expression level of HER2 protein (e.g. HER2(2+) by IHC) and a high amplification of the HER2 gene as defined herein.

In a preferred embodiment of the invention, the modulator of the HER2/neu (ErbB2) signaling pathway is a HER dimerization/signaling inhibitor or an inhibitor of shedding of the HER2 extracellular domain (ECD).

Preferably the HER dimerization/signaling inhibitor is a HER2 dimerization inhibitor. It is also preferred herein that the HER dimerization inhibitor inhibits HER heterodimerization, HER homodimerization, or both.

In a particular preferred embodiment of the present invention the HER dimerization/signaling inhibitor is a HER antibody. The HER antibody may bind to a HER receptor, such as EGFR, HER2 and HER3. Preferably, the antibody binds to HER2. The HER2 antibody may bind to Domain II of HER2 extracellular domain and/or may bind to a junction between domains I, II and III of HER2 extracellular domain.

In another preferred embodiment of the invention, the HER dimerisation inhibitor inhibits heterodimerisation of HER2 with EGFR or HER3 or Her4. In a further preferred embodiment, the HER2 antibody to be employed as a modulator of the HER2 signaling pathway by inhibiting receptor dimerization/signaling in accordance with this invention is Pertuzumab.

Preferably, the HER dimerisation inhibitor is an antibody, preferably the antibody 2C4. Preferred throughout the application is the “antibody 2C4”, in particular the humanized variant thereof (WO 01/00245; produced by the hybridoma cell line deposited with the American Type Culture Collection, Manassass, Va., USA under ATCC HB-12697), which binds to a region in the extracellular domain of HER2 (e.g., any one or more residues in the region from about residue 22 to about residue 584 of HER2, inclusive). Examples of humanized 2C4 antibodies are provided in Example 3 of WO 01/00245. The humanized antibody 2C4 is also called Pertuzumab.

Preferably, the inhibitor of HER shedding is a HER2 shedding inhibitor. It is also preferred herein that the HER shedding inhibitor inhibits HER heterodimerization or HER homodimerization.

In a particular preferred embodiment of the present invention the HER shedding inhibitor is a HER antibody. The HER antibody may bind to a HER receptor, such as EGFR, HER2 and HER3. Preferably, the antibody binds to HER2. Also preferred, the HER2 antibody binds to sub-domain IV of the HER2 extracellular domain (ECD).

In a further preferred embodiment, the HER2 antibody to be employed as a modulator of the HER2 signaling pathway by inhibiting ECD shedding in accordance with this invention is Herceptin/Trastuzumab.

As pointed out herein below, in particular in the medical uses and methods provided herein Herceptin/Trastuzumab is a preferred modulator of the HER2/neu (ErbB2) signaling pathway for the treatment of gastric cancer patients/patient groups as identified by the above recited method and as described herein. This novel gastric cancer patients/patient group is characterized by their biological samples/biopsies which show in vitro tests, using the two biomarkers HER2 status (protein expression level of HER2 and gene amplification status of HER2), an equivocal protein expression level of HER2 (HER2(2+)) and a high amplification status of the HER2 gene (e.g. copy number higher than 4). The terms “equivocal protein expression level of HER2”, and “high amplification status of the HER2 gene” are described herein above. Said semi-quantitative assessment of the expression level of HER2 protein and HER2 gene number may be set in correlation to given control samples which may comprise normal tissue samples, i.e. healthy control samples. Such control samples may be obtained from e.g. healthy volunteers or may be a defined, clearly healthy control tissue from the patient to be assessed for its HER2 gene amplification status and protein expression level. The biological sample to be tested and assessed for said HER2 status/level may in particular be a tissue sample obtained through gastric tissue biopsy or resection.

The term “antibody” herein is used in the broadest sense and specifically covers intact monoclonal antibodies, polyclonal antibodies, multispecific antibodies (e.g., bispecific antibodies) formed from at least two intact antibodies, and antibody fragments, so long as they exhibit the desired biological activity. Also human and humanized as well as CDR-grafted antibodies are comprised.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be constructed as requiring production of the antibody by any particular method. For example, the monoclonal antibodies to be used in accordance with the present invention may be made by the hybridoma method first described by Kohler, G. et al., Nature 256 (1975) 495, or may be made by recombinant DNA methods (see, e.g., U.S. Pat. No. 4,816,567). “Antibody fragments” comprise a portion of an intact antibody. In context of this invention, antibody modulators of the HER2 pathway to be employed in the means and methods provided herein and in particular in the treatment of the newly identified gastric cancer group as defined herein are preferably humanized, fully-human or CDR-grafted antibody molecules. A preferred antibody is Herceptin/Trastuzumab.

The term “sample” shall generally mean any biological sample obtained from an individual/patient. The step of obtaining a sample may be omitted in the methods of the present invention, so that the inventive methods only comprise the evaluation step. Therefore, the present invention relates in one embodiment to an in vitro method for the identification of a patient suspected to suffer from gastric cancer and having an equivocal expression level of HFR2 protein as a responder for or a patient sensitive to a modulator of the HER2/neu (ErbB2) signaling pathway, said method comprising the step of evaluating the gene amplification status of the HER2 gene in a sample, whereby an equivocal expression level of HER2 protein and a high gene amplification status of the HER2 gene is indicative for a responding patient or is indicative for a sensitivity of said patient to said modulator of the HER2/neu (ErbB2) signaling pathway. The sample is preferably a resection sample or a biopsy sample.

The terms “biopsy” and “resection” are well known to those skilled in the art. In context with the present invention, a biopsy sample is a biological sample obtained by removal of single cells or (parts of) tissue from a subject using a needle, a brush, a scraper or a punch, Examples are aspiration biopsy, brush biopsy, core biopsy, vacuum biopsy, core needle biopsy, needle biopsy or punch biopsy. A resection sample is a biological sample obtained by surgical excision or cutting of (parts of) an organ or tissue from a subject using a scalpel, a knife, scissors or other instrument designed for cutting. Examples are gastric tissue resection, comprising at least part of a primary tumour, and a metastatic lesion resection. A biological sample may also comprise circulating tumor cells.

According to the invention, the biological sample may comprise gastric cancer cells and non-gastric cancer cells (other cells). The skilled pathologist is able to differentiate cancer cells from normal gastric tissue cells. Methods for obtaining tissue biopsies, resections and body fluids from mammals are well known in the art.

In one embodiment of the present invention, the novel sub-group of gastric cancer patients as defined herein and being characterized by its biological samples/biopsies showing an equivocal expression level of HER2 protein expression (a level of HER2 (HER2(2+)) and a high gene amplification of the HER2 gene (e.g. an average gene copy number of more than 4 per nucleus/tumor cell), may suffer from invasive gastric cancer, in particular intestinal-type adenocarcinoma, mixed-type adenocarcinoma or diffuse-type adenocarcinoma.

In one embodiment of the present invention, the sample of said patient to be tested in accordance with this invention for its HER2 level/status is obtained before anti-metastatic therapy, i.e. before the treatment with a modulator of the HER2/neu (ErbB2) signaling pathway is initiated. However, also neo-adjuvant or adjuvant therapy and testing of corresponding samples is envisaged.

In a further embodiment of the present invention, a method for the treatment of gastric cancer patients (in particular invasive gastric cancer) is provided, said treatment comprising a step of administering an effective amount of a modulator of the HER2/neu (ErbB2) signaling pathway to a subject identified by the method as provided herein above and a subject in need of such a treatment. Said subject is, in accordance with this invention, preferably a human subject. Biological samples, in particular gastric tissue biopsies/resections of said subject/patient are characterized in having an equivocal expression level of HER2 protein and a high amplification/amplification level of the HER2 gene. As documented herein, the person skilled in the art is readily in a position to determine said HER2 protein expression levels in said biological sample, in particular by immunohistochemical methods known in the art. The same applies, mutatis mutandis, for the determination of the given HER2 amplification/amplification level. Here, as pointed out above, the preferred (but not limiting) determination method is an in situ hybridization technique, like fluorescent in situ hybridization (FISH), chromogenic in situ hybridization (CISH) or silver in situ hybridization (SISH). The patient to be treated in accordance with this invention is preferably a human patient and said biological sample wherein said expression level of HER2 protein and said amplification status of the HER2 gene is determined in vitro is a biological sample from a human patient as described herein.

Again, the gist of the present invention relates to the fact that surprisingly patients suffering from gastric cancer and showing only an equivocal status/level of HER2 protein expression and a high HER2 gene amplification can successfully be treated by a modulator of the HER2/neu (ErbB2) signaling pathway. Said modulator comprises in particular an antibody molecule directed against the HER2 protein. Preferred antibody molecules in this respect are Herceptin/Trastuzumab as well as Pertuzumab (as, inter alia, described in WO 2007/145862).

Other HER2 signaling modulators or HER2 agents to be used in accordance with this invention on patients which show an equivocal expression level of HER2 protein and a high HER2 gene amplification as defined herein, comprise also tyrosine kinase inhibitors, like the oral tyrosine kinase inhibitor Tykerb (lapatinib ditosylate), HKI272 or BIBW229.

As defined hereinabove in context of the inventive in vitro method for the identification of a responder for or a patient sensitive to a modulator of the HER2/neu (ErbB2) signaling pathway, the person skilled in the art can easily deduce with known methods which “HER2 expression status” is present in a biological sample of said patient. The patient to be treated with the herein defined modulator of the HER2/neu (ErbB2) signaling pathway shows in the biological samples an equivocal protein expression level and a high amplification level/status of the HER2 gene. Again, an “equivocal protein expression level” of HER2 corresponds to a 2+ score (“equivocal classification”, see the tables above) in immunohistological tests as described hereinabove and a high HER2 amplification status relates to an average HER2 gene copy number higher than 4 copies of the HER2 gene per tumor cell (for those test systems without an internal centromere control probe) or to a HER2/CEP17 ratio of higher than 2 per copy (for those test systems using an internal chromosome 17 centromere control probe) in a sample obtained from said patient suspected to suffer from or being prone to suffer from a gastric cancer.

The person skilled in the art can also easily detect and/or verify the gene amplification status of the HER2 gene. This is also routinely done by in situ hybridizations, like fluorescence in situ hybridizations (FISH) or bright field in situ hybridizations. Accordingly, when the gene amplification status of HER2 is tested, FISH-tests are routinely used and the read-out may comprise the determination of the average HER2 gene copy number or the so-called HER2/CEP17 ratio, whereby said HER2/CEP17 ratio sets the HER2 signal in relation to a signal obtained with centromere probe (CEP17). In context of the present invention, a new patient group accessible for treatment with the herein defined modulators of the HER2 signaling pathway are gastric cancer patients that show a high gene amplification of HER 2 and an equivocal HER2 protein expression.

In accordance with the present invention, a method of treating preferably a human gastric cancer patient is provided, wherein said patients are evaluated for the expression level of the HER2 protein and for the HER2 gene amplification level, if the expression level of HER2 protein is equivocal. It is envisaged in accordance with this invention that patients showing an “equivocal protein expression level” (corresponding to a 2+score or “equivocal classification”, see the tables above) of HER2 and showing a “high HER2 gene amplification level (preferably a copy number of higher than 4 or a HER2/CEP17 ratio of higher than 2 as defined herein) suspected to suffer from or being prone to suffer from gastric cancer, also invasive gastric cancer, show in accordance with this invention a positive survival benefit, a prolongation in time to progression, and/or show less recurrent gastric cancers when treated with the herein defined modulator(s) of the HER2/neu (ErbB2) signaling pathway, in particular with Herceptin/Trastuzumab.

The medical uses and methods as described herein relate to the use of the herein described modulator of the HER2/neu (ErbB2) signaling pathway, in particular antibodies against or directed to HER2, like and preferably Herceptin/Trastuzumab, on patients that show the herein determined HER2 “equivocal protein expression status” (versus a “low protein expression level” as defined herein above) and that have a “high HER2 gene amplification status as defined herein, e.g. on average more than 4 copies per tumour cell. In context of this invention, said HER2/neu (ErbB2) signaling pathway, in particular antibodies against or directed to HER2, like and preferably Herceptin/Trastuzumab may be employed in anti-metastatic, adjuvant as well as in neo-adjuvant gastric cancer therapies. Accordingly, said “HER2-modulator” may be administered to a patient in need of such a treatment and having the herein defined biomarker status before, during of after a surgical intervention/resection of the cancerous tissue. Therefore, the present invention is useful in anti-metastatic, as well as in neoadjuvant therapy, i.e. the treatment with the herein defined HER2-signalling pathway modulator (like Herceptin/Trastuzumab) given to the herein defined gastric cancer patient group prior to surgery, as well as in adjuvant therapy. Again, the patient group of the present invention to be treated by the means and methods provided herein (in particular with Herceptin/Trastuzumab) are gastric cancer patients wherein the two biomarkers, i.e. HER2 protein expression and HER2 gene amplification status, are assessed and wherein patients are treated having an “equivocal expression status” (HER2(2+)) and a high HER2 gene amplification status is (i.e. higher than 4 copies or in other words at least 5 copies per tumour cell).

In accordance with the medical as well as the diagnostic (in vitro) methods provided herein, the person skilled in the art can, inter alia, determine the status/level of HER2 amplification in a given sample and the expression level/status of HER2 protein by means and methods known in the art. These methods also comprise the comparison of the given sample with a normal control sample, i.e. with a biological sample which is not cancerous and which, inter alia, be derived from a healthy (control) individual or from non-diseased tissue.

Accordingly, the present invention relates to a modulator of the HER2/neu (ErbB2) signaling pathway for use in treating gastric cancer in a patient identified by the method described and defined herein. Also the use of a modulator of the HER2/neu (ErbB2) signaling pathway for the preparation of a pharmaceutical composition for the treatment of gastric cancer in a patient identified by the method of the present invention is envisaged.

In context of the medical embodiments provided herein, i.e. methods and uses, the modulator of the HER2/neu (ErbB2) signaling pathway to be administered to the herein defined patient group (gastric cancer patients/patients with invasive gastric cancer and having equivocal expression level of the HER2 protein (HER2 (2+) and at the same time a “high” HER2 gene amplification status) may be administered as a single anti-tumor agent. However, also co-therapeutic approaches are envisaged and of use, which comprise, inter alia, the administration of further pharmaceuticals, in particular anti-cancer drugs, e.g. in form of a combination therapy. Such an additional therapy may be a chemotherapy and may comprise the administration of drugs like, fluoropyrimidine in combination with cisplatin, anti-metabolite agents (for example gemcitabine), an anti-hormonal compound, a tyrosine kinase inhibitor, a raf inhibitor, a ras inhibitor, a dual tyrosine kinase inhibitor, taxol, a taxane (like paclitaxel or docetaxel), an anthracycline, like doxorubicin or epirubicin, or cisplatin. Also vinorelbine can be used in the inventive co-therapy approaches. Furthermore, co-therapy approaches with in particular Herceptin/Trastuzumab may comprise the administration cyclophosphamide, methotrexate or fluorouracil (which is also known as 5-FU) individual or in form of a combination therapy comprising these three drugs (“CMF therapy”). The combination therapy of a modulator of the HER2/neu (ErbB2) signaling pathway, in particular Herceptin/Trastuzumab with fluoropyrimidine and cisplatin also represents a preferred embodiment of the present invention.

The preferred therapeutic approach provided herein, i.e. the use of a modulator of HER2 signaling may also be combined with another therapy. Such combination therapy may preferably also rely on the use of chemotherapeutic agent, or it may also comprise anti-angiogenic agents which comprise (but are not limited to) the administration of a VEGF blocker, like, e.g. bevacizumab/Avastin or sutent (sunitinib malate-SU-11248).

The person skilled in the art, for example the attending physician, is readily in a position to administer the herein defined modulator of the HER2/neu (ErbB2) signaling pathway to patient/patient group as defined herein. Such an administration may comprise the parenteral route, the oral route, the intravenous route, the subcutaneous route, the intranasal route or the transdermal route. In case of Herceptin/Trastuzumab, the preferred administration route is an intravenous administration. Moreover, the modulator of the HER2/neu (ErbB2) signaling pathway may be administered in an anti-metastatic, neoadjuvant or adjuvant setting. Such an administration of Herceptin/Trastuzumab may, in the novel gastric cancer patients (group)/invasive gastric cancer patients (patient group) as defined herein comprise, inter alia, an administration every day, every other day, every third day, every forth day, every fifth day, once a week, once every second week, once every third week, once every month, etc.

Again, the attending physician may modify, change or amend the administration schemes for modulator of the HER2/neu (ErbB2) signaling pathway in accordance with his/her professional experience. In a particular preferred embodiment of the present invention, a method for the treatment of gastric cancer patient or patient group is provided, said method comprising the administration of Herceptin/Trastuzumab to said patient/patient group, whereby said patient/patient group is characterized in the assessment of a biological sample (in particular a biopsy or resection), said sample showing an equivocal expression level of HER2 protein (HER2(2+)) and a “high HER2 gene amplification status”. Therefore, the present invention also provides for the use of Herceptin/Trastuzumab in the preparation of pharmaceutical compositions for the treatment of gastric cancer patients which are characterized by the herein disclosed biomarker status (an equivocal expression level of HER2 protein and a “high HER2 gene amplification status” as defined herein above) or which have been identified by the herein described in vitro method for the identification of a responder for or a patient sensitive to a modulator of the HER2/neu (ErbB2) signaling pathway. Said gastric cancer patient/patient group may also suffer from an invasive gastric cancer.

The present invention is further described by reference to the following non-limiting figures and examples.

Methods

Study Design

The ToGA trial is a randomized, open-label, multicentre, Phase III study designed to investigate the safety and efficacy of trastuzumab in combination with cisplatin plus a fluoropyrimidine (capecitabine or 5-fluorouracil) versus cisplatin plus a fluoropyrimidine alone in HER2-positive advanced GC.

Patients

Inclusion Criteria:

Histologically confirmed adenocarcinoma of the stomach or gastro-oesophageal junction (GEJ) with inoperable locally advanced or recurrent and/or metastatic disease not amenable to curative therapy

Measurable disease according to the Response Evaluation Criteria in Solid Tumors (RECIST), assessed using imaging techniques (computed tomography or magnetic resonance imaging), or non-measurable evaluable disease—HER2-positive tumour (primary tumour or metastasis) as assessed by the central laboratory. Both IHC and FISH were performed on all patients' samples (either resection or biopsy) in the central laboratory.

An Eastern Cooperative Oncology Group performance status of 0, 1 or 2

Life expectancy of 3 months.

General inclusion criteria:—Male or female, 18 years of age—Signed informed consent.

Assessments

The primary end point is overall survival; secondary end points include progression-free survival, overall response rate and duration of response. For further information on efficacy assessments see abstract LBA4509.10 HER2 testing.

GC tumour samples were formalin fixed and paraffin embedded. The samples (either resection or biopsy) were analyzed at a central laboratory using both IHC (modified HercepTest™) and FISH (pharmDx™; Dako) to determine HER2 status, as recommended by the validation study for HER2 testing in GC. IHC HER2 scoring used the following modified HercepTest™ parameters: staining intensity; complete/incomplete membrane staining; percentage of stained cells; incomplete membrane staining due to lumen/other reason. For the FISH analysis, HER2 positivity was defined as a HER2:CEP17 ratio of 2 or greater. In this study a HER2-positive result was defined as IHC 3+ and/or FISH positive

Results

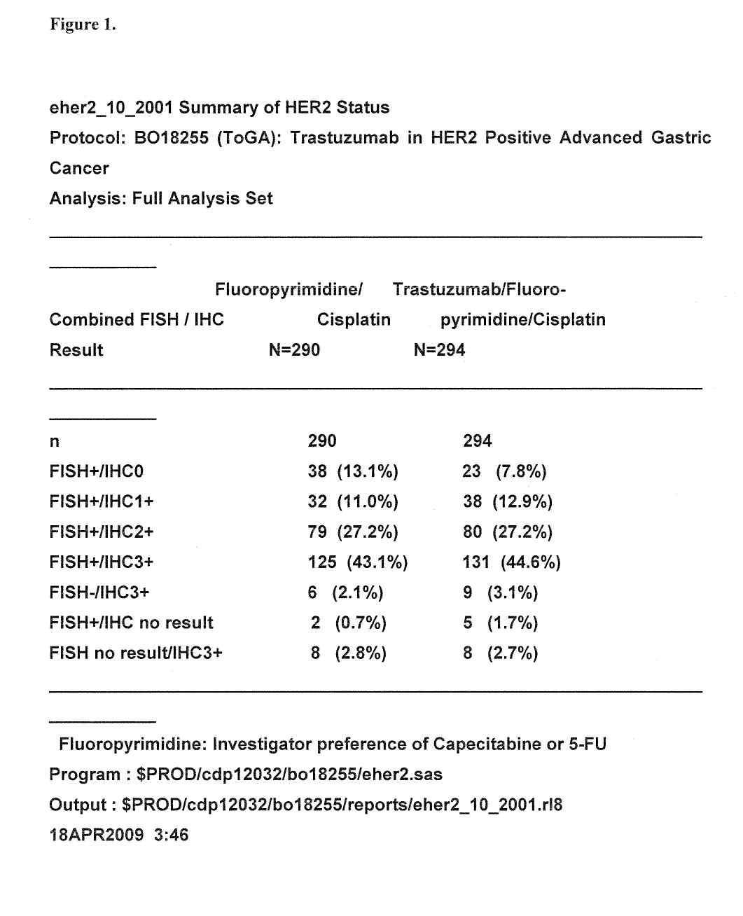

The HER2-screening process in ToGA is now complete: 3807 tumour samples from 24 countries have been assessed for HER2 status in a central laboratory using the modified system described. Of these, 3667 samples were evaluable and 810 have been defined as HER2 positive, giving an overall HER2-positivity rating of 22.1%. An unexpectedly high number of cases were found to be FISH positive/IHC 0/1+ and these were randomized between treatment arms (FIG. 1). In breast cancer HER2 testing, most IHC 0/1+ samples are found to be FISH negative, but in ToGA the frequency of IHC 0/1+/FISH-positive samples was almost as high as IHC 2+/FISH-positive samples (25% vs 28%, respectively). Of the cases that were IHC 3+, 5% were found to be FISH negative.

The HER2-positivity rate was similar between Europe (23.6%) and Asia (23.5%). HER2-positivity rates varied between countries, from 5.9% in Taiwan (n=34) to 32.8% in Australia (n=61).

HER2 positivity varied according to tumour site, with higher rates of HER2 positivity in GEJ (gastroesophageal junction) cancer than in stomach cancer (33.2% vs 20.9% respectively; p<0.001).

In general, the countries with the highest ratio of GEJ:stomach cancer were found to have above-average HER2-positivity rates, although the patient numbers were low. These included France (ratio 0.56; 26.9% HER2 positive), Germany (ratio 0.53; 23.7% HER2 positive) and the UK (ratio 0.33; 25.8% HER2 positive). Similarly, there was a significant difference in HER2 positivity (p<0.001) based on histological subtype (Lauren classification): 32.2% in intestinal vs 6.1/20.4% in diffuse/mixed cancer. Again, countries with higher ratios of intestinal: diffuse/ mixed cancer had increased HER2-positivity rates, such as the UK (ratio 3.4; 25.8% HER2 positive), Australia (ratio 2.6; 32.8% HER2 positive), Japan (ratio 2.8; 28.1% HER2 positive) and Portugal (ratio 3.33; 22.4% HER2 positive).

The ToGA trial is the first Phase III trial to provide information on the incidence of HER2 positivity in a prospective manner in advanced GC. The ToGA screening program observed a HER2-positivity rate of 22.1% in advanced GC, which is comparable to rates previously observed in breast cancer. Data also show that variations in HER2 positivity in GC across countries can be explained by differences in histological subtype and tumour site. Gastric tumours tend to be much more heterogeneous than breast tumours, hence HER2 testing in GC is different to that in breast cancer. Comparisons of this screening data against ToGA efficacy has lead to the novel treatment algorithm as disclosed in the present invention

The Figures show:

FIG. 1.

3807 tumour samples from the ToGA study from 24 countries have been assessed for HER2 status in a central laboratory using the modified system described in the Table above. Of these, 584 patients were included to the study.

FIG. 2.

Hazard Ratio (HR) and CI for HER2 subgroups: overall survival (OS)

FIG. 3.

Hazard Ratio (HR) and CI for HER2 subgroups: progression free survival (PFS)

1. An in vitro method for the identification of a patient suspected to suffer from gastric cancer and having an equivocal expression level of HER2 protein as a responder for or a patient sensitive to a modulator of the HER2/neu (ErbB2) signaling pathway, said method comprising the following steps:
(a) obtaining a sample from said patient; and
(b) evaluating
the gene amplification status of the HER2 gene in said sample,
whereby an equivocal expression level of HER2 protein and a high gene amplification status of the HER2 gene is indicative for a responding patient or is indicative for a sensitivity of said patient to said modulator of the HER2/neu (ErbB2) signaling pathway.

2. The method of claim 1, wherein said protein expression level of HER2 is or was determined by an immunohistochemical (IHC) method.

3. The method of claim 1 or 2, wherein said equivocal protein expression level of HER2 is HER2(2+), as determined in a biopsy sample.

4. The method of claim 1 or 2, wherein said equivocal protein expression level of HER2 is HER2(2+), as determined in a resection sample.

5. The method of claim 1 or claim 2, wherein said gene amplification status of the HER2 gene is detected by an in situ hybridization (ISH) method.

6. The method of claim 1, wherein said high amplification status of the HER2 gene is an average gene copy number of the HER2 gene of higher than 4 or an average gene copy number of HER2 equal to or higher than 2 per chromosome 17 copy.

7. The method of claim 1, wherein the response rate of a group of patients identified by the method of any of claims 1 to 6 to a modulator of the HER2/neu (ErbB2) signaling pathway is at least 20%.

8. The method of claim 1, wherein said sample is selected from the group consisting of gastric tissue resection, gastric tissue biopsy, tissue from a metastatic lesion resection and circulating tumor cells.

9. The method of claim 2, wherein said immunohistochemical method is performed with the HerceptTest™ assay.

10. The method of claim 5, wherein said in situ hybridization is selected from the group consisting of fluorescent in situ hybridization (FISH), chromogenic in situ hybridization (CISH) and silver in situ hybridization (SISH).

11. The method of claim 10, wherein said FISH test is selected from the group consisting of “Inform®” kit, “Pathvysion™” kit or “pharmDx™” kit.

12. The method of claim 10, wherein said CISH test is selected from the group consisting of SPoT-Light® HER2 CISH™ and ZytoDot® SPEC HER2 Probe kit.

13. The method of claim 10, wherein said SISH test is the “Inform™” HER2 DNA probe in combination with the ultraView™ SISH detection kit

14. The method of claim 1, wherein said sample is obtained before anti-metastatic, neoadjuvant or adjuvant therapy.

15-16. (canceled)

17. A method for the treatment of gastric cancer comprising administering an effective amount of a modulator of the HER2/neu (ErbB2) signaling pathway to a subject identified by the method of claim 1 in need of such a treatment.

18. The method of claim 17, wherein said subject is a human.

19. The method of claim 17 or 18, wherein said modulator of the HER2/neu (ErbB2) signaling pathway is administered as a single anti-tumor agent.

20. The method of claim 17 or 18, wherein said modulator of the HER2/neu (ErbB2) signaling pathway is administered in form of a combination therapy.

21. The method of claim 20, wherein the therapy used in said combination therapy is chemotherapy.

22. The method of claim 21, wherein said chemotherapy is selected from the group consisting of fluoropyrimidine in combination with cisplatin, anthracycline/taxane chemotherapy, therapy with an anti-metabolite agent, therapy with an anti-hormonal compound, therapy with a tyrosine kinase inhibitor, therapy with a raf inhibitor, therapy with a ras inhibitor, therapy with a dual tyrosine kinase inhibitor, therapy with taxol, therapy with taxane, therapy with doxorubicin, therapy with adjuvant (anti-) hormone drugs, and therapy with cisplatin.

23. The method of claim 17, wherein said modulator of the HER2/neu (ErbB2) signaling pathway is administered by any one of a parenteral route, oral route, intravenous route, subcutaneous route, intranasal route or transdermal route.

24. The method of claim 17, wherein said modulator of the HER2/neu (ErbB2) signaling pathway is administered in an anti-metastatic, neoadjuvant or adjuvant setting.

25. The method of claim 17, wherein said modulator of the HER2/neu (ErbB2) signaling pathway is a HER dimerization/signaling inhibitor or an inhibitor of HER shedding.

26. The method of claim 25, wherein said HER dimerization inhibitor is a HER2 dimerization inhibitor.

27. The method of claim 25 or 26, wherein said HER dimerization inhibitor inhibits HER heterodimerization or HER homodimerization.

28. The method of claim 25 or claim 26, wherein said HER dimerization inhibitor is a HER antibody.

29. The method of claim 28, wherein said HER antibody binds to a HER receptor selected from the group consisting of EGFR, HER2 and HER3.

30. The method of claim 29, wherein said antibody binds to HER2 .

31. The method of claim 30, wherein said HER2 antibody binds to domain II of HER2 extracellular domain.

32. The method of claim 31, wherein said antibody binds to a junction between domains I, II and III of HER2 extracellular domain.

33. The method of claim 31 or claim 32, wherein said HER2 antibody is Pertuzumab.

34. The method of claim 25, wherein said inhibitor of HER shedding is a HER2 shedding inhibitor.

35. The method of claim 25 or claim 34, wherein said inhibitor of HER shedding inhibits HER heterodimerization or HER homodimerization.

36. The method of claim 25 or claim 34, wherein said inhibitor of HER shedding is a HER antibody.

37. The method of claim 36, wherein said HER antibody binds to a HER receptor selected from the group consisting of EGFR, HER2 and HER3.

38. The method of claim 37, wherein said antibody binds to HER2.

39. The method of claim 38, wherein said HER2 antibody binds to sub-domain IV of the HER2 extracellular domain.

40. The method of any one of claims 37 to 39, wherein said HER2 antibody is trastuzumab.

41. The method of claim 1 or claim 17, wherein said gastric cancer is invasive gastric cancer.

42. The method of claim 41, wherein said gastric cancer is selected from the group consisting of intestinal-type adenocarcinoma, mixed-type adenocarcinoma and diffuse-type adenocarcinoma.
